(19) United States
(12) Patent Application Publication
(10) Pub. No.: US 2019/0388558 A1
(43) Pub. Date: Dec. 26, 2019

(54) KARTOGENIN DERIVATIVE-CONTAINING POLYMERIC MICELLE, HYALURONIC ACID HYDROGEL, METHOD FOR PRODUCING THE SAME, AND USE THEREOF

(71) Applicant: DONGGUK UNIVERSITY INDUSTRY-ACADEMIC COOPERATION FOUNDATION, Seoul (KR)

(72) Inventors: GUN IL IM, Seoul (KR); MI LAN KANG, Seoul (KR)

(21) Appl. No.: 16/174,902

(22) Filed: Oct. 30, 2018

(30) Foreign Application Priority Data
Jun. 20, 2018 (KR) 10-2018-0070834

(51) Int. Cl. A61K 47/69; A61K 31/728; A61K 47/60; A61K 9/00; A61P 19/02

(52) U.S. Cl. CPC A61K 47/6907; A61K 31/728; A61P 19/02; A61K 47/6903; A61K 9/0019; A61K 47/60

(57) ABSTRACT

The present invention relates to a polymeric micelle including a kartogenin derivative, a hyaluronic acid hydrogel including the same, a method for producing the same, and a use thereof, and the polymeric micelle and the hyaluronic acid derivative hydrogel including the polymeric micelle slowly release kartogenin, and thus may be usefully used for the purpose of preventing or treating various cartilage disorder-related diseases such as degenerative arthritis because an effect of regenerating chondrocytes while protecting chondrocytes is excellent.

### CROSS-REFERENCE TO RELATED APPLICATIONS

This U.S. non-provisional application claims priority under 35 U.S.C § 119 to Korean Patent Application No. 10-2018-0070834, filed on Jun. 20, 2018, in the Korean Intellectual Property Office, the entire disclosure of which is hereby incorporated by reference herein.

### TECHNICAL FIELD

The present invention relates to a kartogenin derivative-containing polymeric micelle, a hyaluronic acid hydrogel, a method for producing the same, and a use thereof.

### BACKGROUND ART

Degenerative arthritis is a chronic disorder accompanied by inflammation and pain caused by damage to the bones, ligaments, and the like due to progressive damage or degenerative changes in the cartilage that protects joints, and is a representative senile and degenerative disorder experienced by about 85% of the elderly population aged 65 years and over. Recently, the number of patients with degenerative arthritis has been rapidly increasing by 5% or more each year even under the age of 55 due to an increase in obese patients caused by a westernized diet, surgical joint damage, immoderate joint movement, and genetic problems, and patients in the workable age group have become a socially big problem, such as the restriction of economic activity due to severe chronic joint pain.

As a pathological cause for degenerative arthritis, it has been reported that a natural aging process and menopause are closely related, but since clear pathological causes are unknown, it is difficult to develop a clinical therapeutic agent for treating degenerative arthritis. Accordingly, the clinical treatment for degenerative arthritis currently focuses on relief of chronic joint pain, and a physical therapy for degenerative joints, joint cleaning technique using anti-pain drugs, and artificial joint replacement surgery have been a mainstream of the clinical treatment. However, since these medical treatments provide only a temporary joint pain relief effect, a repeated treatment is required, and in the case of artificial joint replacement surgery, there exists a problem in that the surgery is restrictively performed according to the age and the patient's medical history.

In order to overcome this problem, studies of transplanting stem cells have been actively conducted, but since the survival of transplanted mesenchymal stem cells cannot be guaranteed and the differentiation into chondrocytes in vivo after transplantation and the cartilage tissue formation efficiency are not proved, it is difficult to expect the transplantation of stem cells as a universal treatment method. In order to overcome the problems of in vivo distribution and differentiation of cell therapeutic agents, techniques for delivering cells by using various forms of biomaterials as scaffolds have been used, and furthermore, techniques for manufacturing a 3-dimensional structured artificial cartilage tissue (tissue engineered cartilage) in vitro have been developed. However, an existing scaffold in the form of a hydrogel has insufficient cell viability and cartilage differentiation rate, a scaffold in the form of a membrane cannot form 3-dimensional cartilage tissues, and when a scaffold in the form of a 3-dimensional sponge or mesh is used, the bonding force between a manufactured artificial cartilage and an original tissue (host tissue) is low and thus the cartilage regeneration rate is insufficient.

Thus, the present inventors have conducted intensive studies in order to discover a material for improving degenerative arthritis and a method for producing the same, and as a result, the present inventors confirmed that a polymeric micelle including a kartogenin derivative and a hyaluronic acid derivative hydrogel have excellent cell viability and chondrocyte regeneration effect, thereby completing the present invention.

### REFERENCES OF THE RELATED ART

### Patent Document

(Patent Document 1) Korean Patent No. 10-1593318

### SUMMARY OF THE INVENTION

An object of the present invention is to provide a polymeric micelle including a PEGylation product of a polyethylene glycol derivative and a kartogenin derivative.

Another object of the present invention is to provide a hyaluronic acid derivative hydrogel including a polymeric micelle.

Still another object of the present invention is to provide a composition for protecting or regenerating cartilage, including a hyaluronic acid derivative hydrogel.

Yet another object of the present invention is to provide a pharmaceutical composition for preventing or treating degenerative arthritis, including a hyaluronic acid derivative hydrogel.

Still yet another object of the present invention is to provide a method for producing a polymeric micelle.

A further object of the present invention is to provide a method for producing a hyaluronic acid derivative hydrogel.

An exemplary embodiment of the present invention provides a polymeric micelle including a PEGylation product of a polyethylene glycol derivative and a kartogenin derivative, in which the polyethylene glycol derivative is a polyethylene glycol derivative in which one of the end hydroxyl groups of polyethylene glycol is substituted with an amine group.

The PEGylation product of the polyethylene glycol derivative and the kartogenin derivative according to a specific exemplary embodiment of the present invention may form a self-assembled micelle, and hydrophobic kartogenin is positioned at the core of the micelle, and thus may be released sustainably in vivo.

The term “PEGylation” used in the present invention means to covalently or non-covalently bond or fuse polyethylene glycol or a polyethylene glycol derivative to a target material. The PEGylation may be achieved by allowing a reactive derivative of polyethylene glycol to react with the target molecule.

In the present invention, the “kartogenin” is a compound that allows chondrocytes to be produced by triggering the activity of mesenchymal stem cells present in the cartilage and promotes the regeneration of damaged cartilage.

A kartogenin derivative according to a specific exemplary embodiment of the present invention may be a compound having an ester group produced by condensation of a compound including a hydroxyl group with a carboxyl group of kartogenin, and may include both an ester group and a carboxyl group.

The PEGylation may be achieved by a reaction of a primary amine group of a polyethylene glycol derivative with a carboxyl group of a kartogenin derivative, and a PEGylation product produced by the reaction may include a secondary amine group.

The PEGylation product according to a specific exemplary embodiment of the present invention may have a chain structure which has a hydrophilic part such as a carboxyl group at one end and has hydrophobic kartogenin at the other end. That is, the PEGylation product has amphiphilic properties, and thus may be self-assembled to maintain a micelle structure, and hydrophobic kartogenin is positioned at the core part of the micelle under the polar conditions.

According to a specific exemplary embodiment of the present invention, the polyethylene glycol derivative may be a compound represented by the following Formula 1.

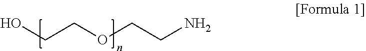

In Formula 1, n may be an integer from 1 to 100. When n is larger than 100, it is difficult to form and maintain particles in a hydrogel, and preferably, n may be 45. The compound represented by Formula 1 may have about 2 KDa.

According to a specific exemplary embodiment of the present invention, the PEGylation product may be a compound represented by the following Formula 2.

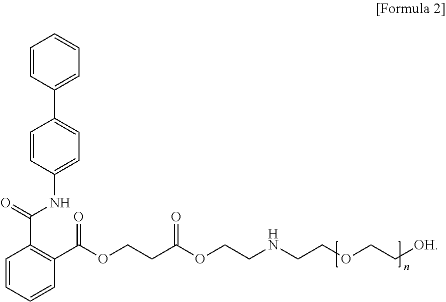

In Formula 2, n may be an integer from 1 to 100.

According to a specific exemplary embodiment of the present invention, the PEGylation product may be a compound represented by the following Formula 3.

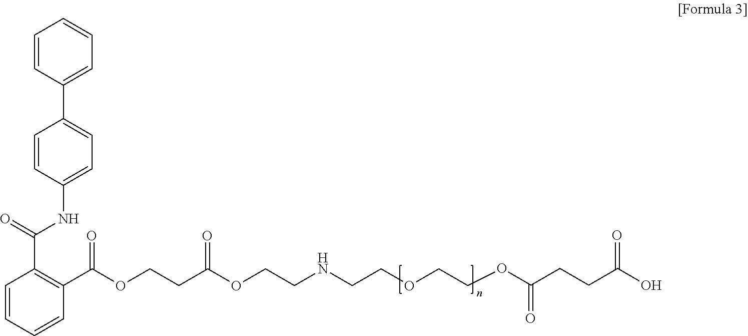

In Formula 3, n may be an integer from 1 to 100.

Another exemplary embodiment provides a hyaluronic acid derivative hydrogel, in which the hydrogel includes a polymeric micelle including a PEGylation product of a polyethylene glycol derivative in which one of the end hydroxyl groups of polyethylene glycol is substituted with an amine group and a kartogenin derivative, and the hyaluronic acid derivative is formed by condensation of a carboxyl group of hyaluronic acid with an amine group of alkylene diamine.

Since the polymeric micelle according to a specific exemplary embodiment of the present invention may be self-assembled under the polar conditions to form a micelle structure, the release of hydrophobic kartogenin may be delayed, but when the micelle is used by being included in a hyaluronic acid derivative hydrogel, the release of kartogenin may be further delayed by a covalent bond of the kartogenin derivative and the hyaluronic acid derivative, so that the cytotoxicity is further reduced and the cartilage regeneration effect and the like are excellent. Further, the hyaluronic acid derivative hydrogel including the polymeric micelle has a much better cartilage regeneration effect, and the like than a free hyaluronic acid hydrogel.

In a specific exemplary embodiment of the present invention, the alkylene diamine may be ethylene diamine.

Before hyaluronic acid (HA) and polyethylene glycol (PEG)/kartogenin (KGN) are crosslinked to each other, when HA and ethylene diamine (EDA) are allowed to react with each other to form HA-EDA, the breakdown of hyaluronic acid may be significantly reduced, and a covalent bond between HA and PEG/KGN may be promoted.

The hyaluronic acid derivative according to a specific exemplary embodiment of the present invention may be a hyaluronic acid derivative in which one of the functional groups of hyaluronic acid may be modified with an amine group by a reaction with alkylene diamine, and for example, may be a hyaluronic acid derivative in which a carboxyl group of hyaluronic acid is modified with an amine group by a condensation reaction with ethylene diamine.

According to a specific exemplary embodiment of the present invention, the polymeric micelle may contain a compound represented by the following Formula 2.

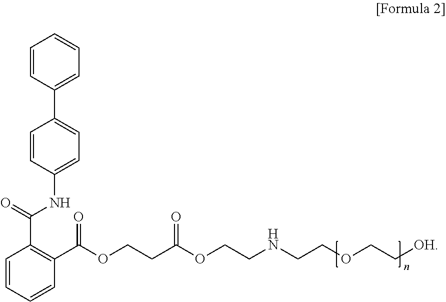

According to a specific exemplary embodiment of the present invention, the polymeric micelle may contain a compound represented by the following Formula 3.

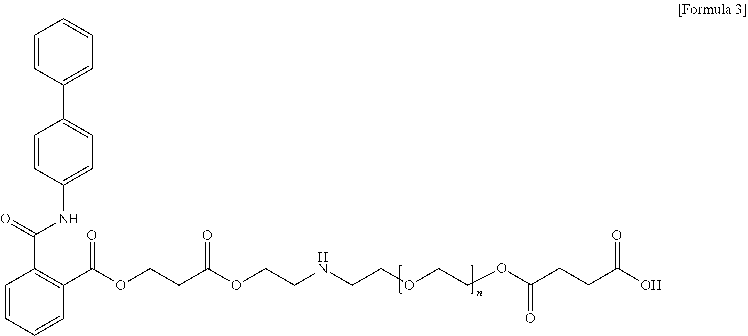

According to a specific exemplary embodiment of the present invention, the hyaluronic acid derivative may be a compound represented by the following Formula 4.

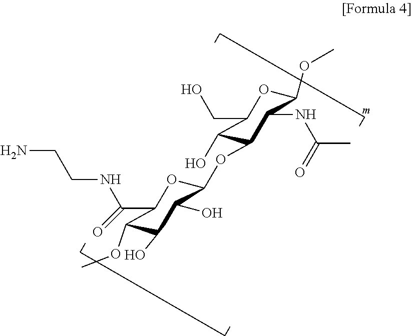

In Formula 4, m may be an integer from 100 to 3,000. When m is larger than 3,000, the stiffness is so high that the use of the compound as an injectable agent is not easy, and when m is smaller than 100, the stiffness is so low that the use of the compound as a therapeutic agent for arthritis is inappropriate. The compound represented by Formula 1 may have about 1,000 KDa.

According to a specific exemplary embodiment of the present invention, the polymeric micelle may be completely or partially covalently bonded to the hyaluronic acid derivative.

According to a specific exemplary embodiment of the present invention, the polymeric micelle may be covalently bonded to a hyaluronic acid derivative while maintaining a micelle structure in a hyaluronic acid derivative hydrogel. Accordingly, a part or all of the polymeric micelle of the present invention may be present while being covalently bonded to a hyaluronic acid derivative of the present invention in a hydrogel.

According to a specific exemplary embodiment of the present invention, the covalent bond may be formed by a dehydration condensation reaction of a carboxyl group derived from the polymeric micelle with an amine group derived from the hyaluronic acid derivative.

According to a specific exemplary embodiment of the present invention, the hyaluronic acid hydrogel may include a compound represented by the following Formula 5.

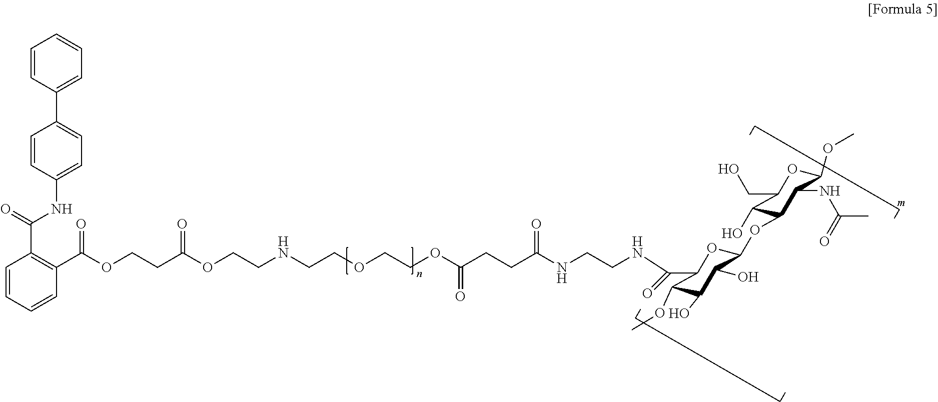

In Formula 5, n may be an integer from 1 to 100, and m may be an integer from 100 to 3,000.

Yet another exemplary embodiment provides a composition for protecting or regenerating cartilage, including a hyaluronic acid derivative hydrogel formed by condensation of a carboxyl group of hyaluronic acid with an amine group of alkylene diamine, which includes a polymeric micelle including a PEGylation product of a polyethylene glycol derivative, in which one of the end hydroxyl groups of polyethylene glycol is substituted with an amine group, and a kartogenin derivative.

When the polymeric micelle according to a specific exemplary embodiment of the present invention is included in a hyaluronic acid derivative hydrogel, the in vivo release of kartogenin may be further delayed by a covalent bond of a hydrophilic part of the micelle with a hyaluronic derivative. Accordingly, since the cytotoxicity is remarkably low and the hyaluronic acid derivative hydrogel thickens the cartilage, there is an effect of protecting the cartilage. In addition, since the hyaluronic acid derivative hydrogel has a remarkably better effect of promoting the regeneration of chondrocytes than a free hyaluronic acid hydrogel, the hyaluronic acid derivative hydrogel may be usefully used for preventing or treating various cartilage-related diseases such as degenerative arthritis.

The term “cartilage” used in the present invention refers to hyaline cartilage, fibrocartilage, and/or elastic cartilage, and the like, and includes all the cartilage sites such as articular cartilage, ear cartilage, nasal cartilage, elbow cartilage, meniscus, knee cartilage, costal cartilage, ankle cartilage, tracheal cartilage, laryngeal cartilage, and vertebrae cartilage.

The term “cartilage protection” used in the present invention refers to the increase in the thickness of cartilage, and includes the delay or maintenance of the decrease in the existing cartilage thickness or the prevention or delay of the deterioration in the pathological state of the existing cartilage.

The term “cartilage regeneration” used in the present invention means that the cartilage is regenerated when transplanted into cartilage defects or damaged parts, and includes the appearance of effects of improving or treating the cartilage damage.

According to a specific exemplary embodiment of the present invention, the hyaluronic acid derivative hydrogel may contain a compound represented by the following Formula 5.

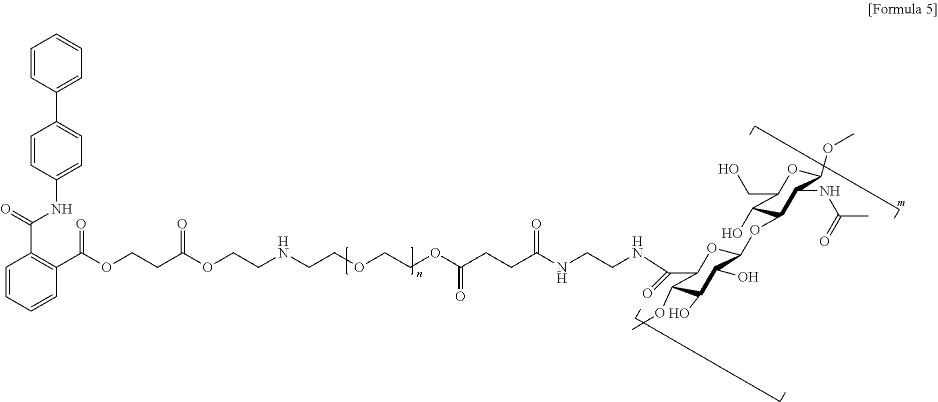

Still another exemplary embodiment provides a pharmaceutical composition for preventing or treating degenerative arthritis, including a hyaluronic acid derivative hydrogel formed by condensation of a carboxyl group of hyaluronic acid with an amine group of alkylene diamine, which includes a polymeric micelle including a PEGylation product of a polyethylene glycol derivative, in which one of the end hydroxyl groups of polyethylene glycol is substituted with an amine group, and a kartogenin derivative.

Since degenerative arthritis is accompanied by inflammation and pain caused by damage to the bones, ligaments, and the like due to progressive damage or degenerative changes in the cartilage that protects joints, symptoms of degenerative arthritis may be improved by inducing the differentiation of chondrocytes.

The pharmaceutical composition according to a specific exemplary embodiment of the present invention may include a pharmaceutically acceptable carrier. A pharmaceutically acceptable carrier included in the pharmaceutical composition is typically used in formulation, and includes lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, and the like, but is not limited thereto. The pharmaceutical composition may additionally include a lubricant, a wetting agent, a sweetening agent, a flavoring agent, an emulsifier, a suspending agent, a preservative, and the like, in addition to the aforementioned ingredients. Suitable pharmaceutically acceptable carriers and formulations are described in detail in Remington's Pharmaceutical Sciences (22th ed., 2013).

The pharmaceutical composition according to a specific exemplary embodiment of the present invention may further include one or more materials exhibiting the activity of preventing or treating degenerative arthritis. In addition, the pharmaceutical composition according to a specific exemplary embodiment of the present invention may be used alone or in combination with methods of using surgery, hormone treatment, drug treatment and/or biological response modifiers for treating degenerative arthritis.

The pharmaceutical composition according to a specific exemplary embodiment of the present invention may include various base materials and/or additives required and appropriate for the formulation of a dosage form thereof, and may be produced by further including a publicly-known compound such as a nonionic surfactant, a silicone polymer, an extender pigment, a fragrance, an antiseptic agent, a disinfectant, an oxidation stabilizer, an organic solvent, an ionic or nonionic thickener, a softener, an antioxidant, a free radical destruction agent, an opacifier, a stabilizer, an emollient, silicone, α-hydroxy acid, an antifoaming agent, a moisturizer, a vitamin, an insect repellent, a preservative, a surfactant, an anti-inflammatory agent, a substance P antagonist, a filler, a polymer, a propellant, a basic or acidic agent, or a coloring agent within a range that does not degrade the effect thereof.

The pharmaceutical composition according to a specific exemplary embodiment of the present invention may be administered parenterally, and may be applied, for example, by injection.

An adequate administration amount of the pharmaceutical composition according to a specific exemplary embodiment of the present invention may be prescribed variously depending on factors, such as formulation method, administration method, age, body weight, gender or disease condition of the patient, diet, administration time, administration route, elimination rate, and response sensitivity. A preferred administration amount of the pharmaceutical composition according to a specific exemplary embodiment of the present invention is within a range of 0.001 to 1,000 mg/kg based on an adult.

According to a specific exemplary embodiment of the present invention, the hyaluronic acid derivative hydrogel may contain a compound represented by the following Formula 5.

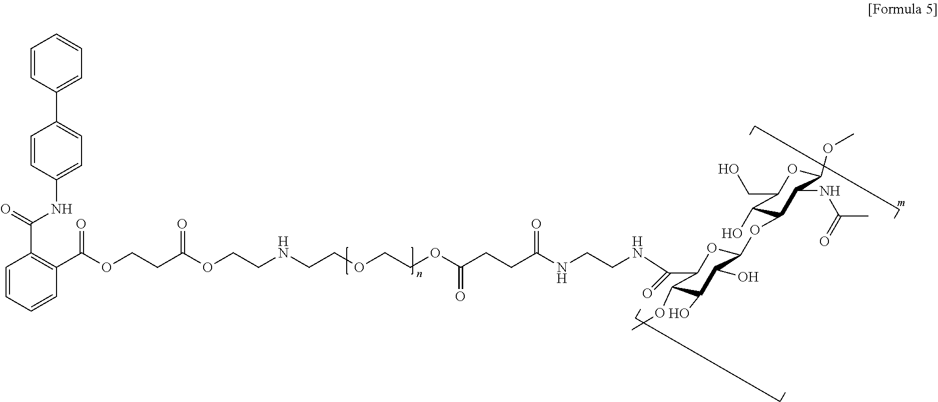

According to a specific exemplary embodiment of the present invention, the composition may be an injectable dosage form.

Still yet another exemplary embodiment provides a method for producing a polymeric micelle, the method including:

producing a compound represented by the following Formula 8, in which an ester group is formed by allowing kartogenin represented by the following Formula 6 and 3-hydroxypropionic acid represented by the following Formula 7 to react with each other;

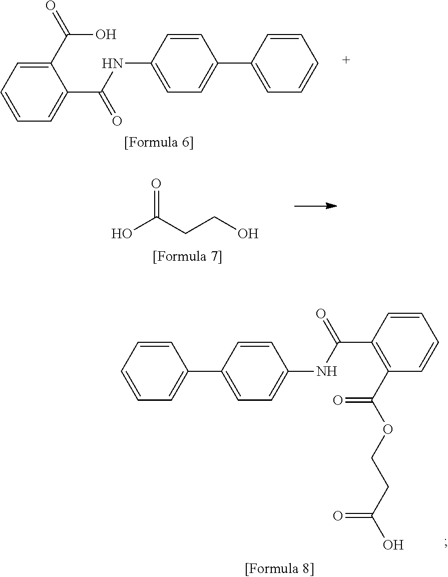 [SMILES: O=C(O)c1ccccc1C(=O)Nc1ccc(-c2ccccc2)cc1 ; O=C(O)CCO ; O=C(O)CCOC(=O)c1ccccc1C(=O)Nc1ccc(-c2ccccc2)cc1]

producing a compound represented by the following Formula 2, in which a secondary amine group is formed by allowing a compound represented by the following Formula 8 and a compound represented by the following Formula 1 to react with each other

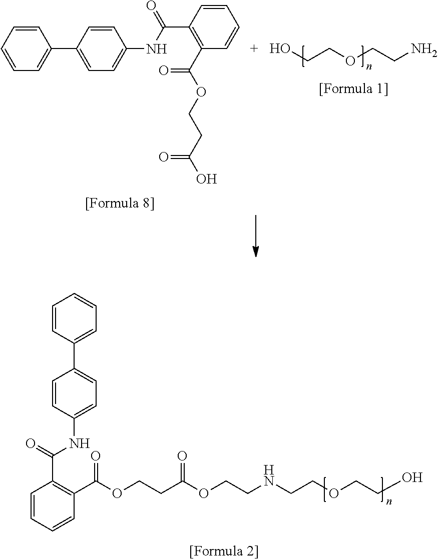

(n is an integer from 1 to 100); and

producing a compound represented by the following Formula 3, into which a carboxyl group is introduced by allowing a compound represented by the following Formula 2 and succinic anhydride to react with each other

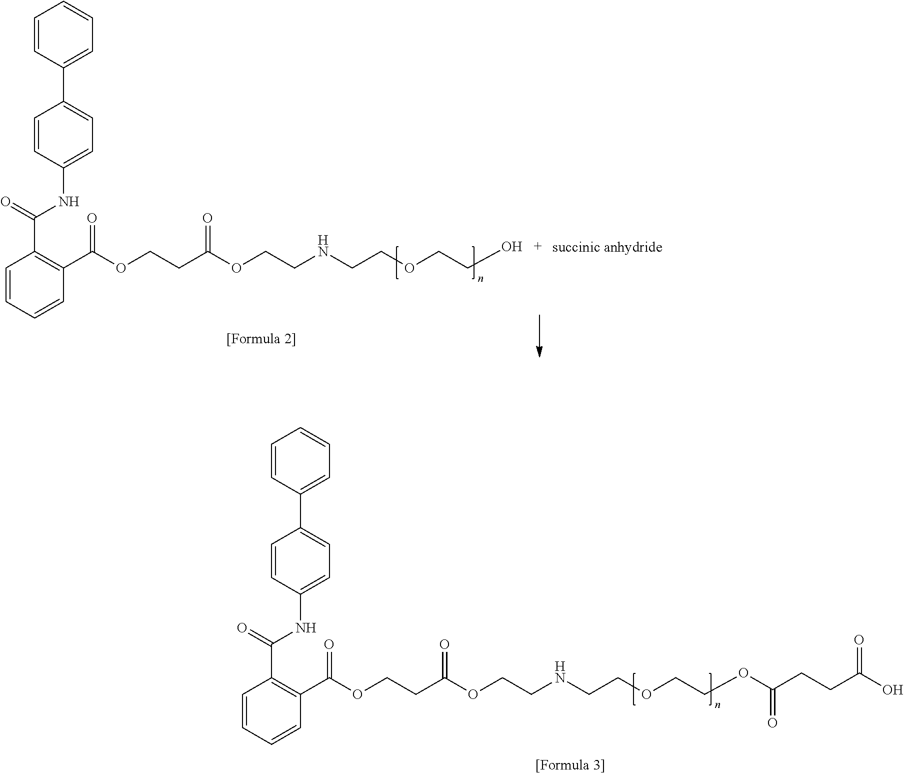

(n is an integer from 1 to 100).

By the method for producing a polymeric micelle according to a specific exemplary embodiment of the present invention, an ester group is formed by allowing a carboxyl group of kartogenin and a hydroxyl group of 3-hydroxypionic acid to react with each other and a compound represented by Formula 2 is produced by PEGylation, and then it is possible to produce a compound represented by Formula 3, in which a hydroxyl group is modified with a carboxyl group by allowing the produced compound and succinic anhydride to react with each other.

By the method for producing a polymeric micelle according to a specific exemplary embodiment of the present invention, it is possible to produce a polymeric micelle formed by the self-assembly of an amphiphilic polymer and an amphiphilic polymer.

A further exemplary embodiment provides a method for producing a hyaluronic acid derivative hydrogel, the method including:

producing a hyaluronic acid derivative represented by the following Formula 4, in which an amide group is formed by allowing hyaluronic acid represented by the following Formula 9 and ethylene diamine to react with each other

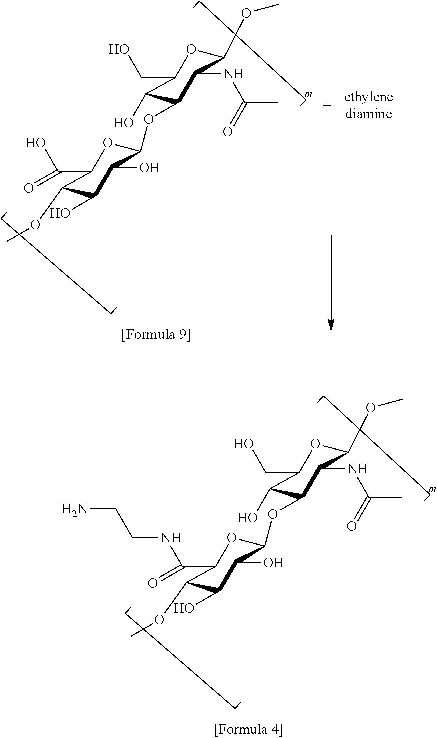

(m is an integer from 100 to 3,000); and

producing a hyaluronic acid derivative hydrogel including a compound represented by the following Formula 5 by allowing the polymeric micelle produced by the production method according to a specific exemplary embodiment of the present invention and the compound represented by Formula 4 to react with each other

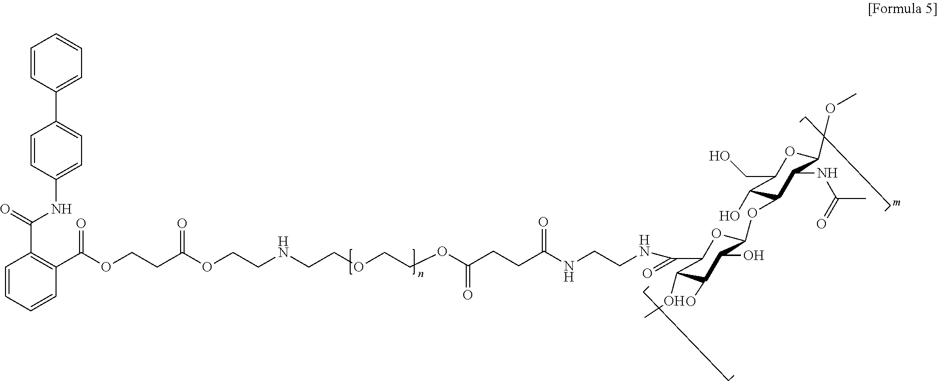

(n is an integer from 1 to 100, and m is an integer from 100 to 3,000).

By the method for producing a hyaluronic acid derivative hydrogel according to a specific exemplary embodiment of the present invention, a carboxyl group of hyaluronic acid is modified with an amine group, and then the compound represented by Formula 5 may be produced by condensation reaction of a unit molecule constituting the polymeric micelle produced by the production method according to a specific exemplary embodiment of the present invention with a hyaluronic acid derivative in which a carboxyl group is modified with amine.

According to the method for producing a hyaluronic acid derivative hydrogel according to a specific exemplary embodiment of the present invention, since HA is allowed to react with EDA before HA and PEG/KGN are crosslinked to each other, the breakdown of hyaluronic acid may be significantly reduced, and a covalent bond between HA and PEG/KGN may be promoted.

A polymeric micelle including the kartogenin derivative of the present invention and a hyaluronic acid derivative hydrogel including the polymeric micelle may slowly release kartogenin, and thus may be usefully used for the purpose of preventing or treating various cartilage disorder-related diseases such as degenerative arthritis because an effect of regenerating chondrocytes while protecting chondrocytes is excellent.

### BRIEF DESCRIPTION OF THE DRAWINGS

FIG. 1 is a schematic view illustrating a process of producing a polymeric micelle (SG-PEG/KGN) according to a specific exemplary embodiment of the present invention and a hyaluronic acid derivative hydrogel (HA/PEG/KGN) including the polymeric micelle.

FIG. 2 is a schematic view illustrating a process (hydrolysis) in which kartogenin is released from a micelle (PEG-KGN-micelle) formed by the polymeric micelle according to a specific exemplary embodiment of the present invention and a micelle included in the hydrogel (HA/PEG/KGN).

FIG. 3 is a graph illustrating the critical micelle concentration (CMC, 0.315 mg/ml) of PEG/KGN.

FIG. 4 illustrates FTIR spectrum results of kartogenin (KGN) and KGN-COOH for C—O, C═O and —CH functional groups.

FIG. 5 illustrates FTIR spectra of KGN—COOH, OH-PEG-HN₂, ON-PEG/KGN, and SA-PEG/KGN for C═C, C═O and C—H functional groups.

FIG. 6 illustrates each functional group of KGN—COOH, OH-PEG-HN₂, and SA-PEG/KGNH by ¹H NMR results.

FIG. 7 illustrates FTIR spectra of SA-PEG/KGN, HA-EDA, and HA/PEG/KGN for C═C, C—H and O—H functional groups.

FIG. 8 illustrates each functional group of SA-PEG/KGN by ¹H NMR results.

FIG. 9 illustrates FTIR spectra of hyaluronic acid (HA) and HA-EDA for an N—H functional group.

FIG. 10 illustrates each functional group of hyaluronic acid (HA) and HA-EDA by ¹H NMR results.

FIG. 11 illustrates each functional group of HA/PEG/KGN by ¹H NMR results.

FIG. 12 illustrates each functional group of HA-EDA by ¹H NMR results.

FIG. 13 illustrates SEM photographs of PEG/KGN micelle (A), HA/PEG/KGN hydrogel (B) and lyophilized HA/PEG/KGN hydrogel (C) in water, and is a graph illustrating the diameters of PEG/KGN micelle (D) and PEG/KGN micelle (E) in the HA/PEG/KGN hydrogel.

FIG. 14 is a graph illustrating cumulative release amounts of KGN from the PEG/KGN micelle or the HA/PEG/KGN hydrogel.

FIG. 15 is a graph illustrating a deterioration analysis result of the HA/PEG/KGN hydrogel.

FIG. 16 is a graph illustrating cytotoxicity of the HA/PEG/KGN hydrogel.

FIG. 17 is a set of fluorescent photographs illustrating a live/dead analysis result using the HA/PEG/KGN hydrogel.

FIG. 18 is a set of fluorescent photographs comparing effects of protecting cartilage of a normal, a vehicle, kartogenin (KGN), hyaluronic acid (HA) and an HA/PEG/KGN hydrogel in a rat model through Safranin-O staining, COL2 immunohistochemistry or immunohistochemistry for aggrecan.

FIG. 19 is a graph illustrating OARSI scores (A) and Mankin scores (B) of rat models into which a normal, a vehicle, kartogenin (KGN), hyaluronic acid (HA), and an HA/PEG/KGN hydrogel are injected in a rat model.

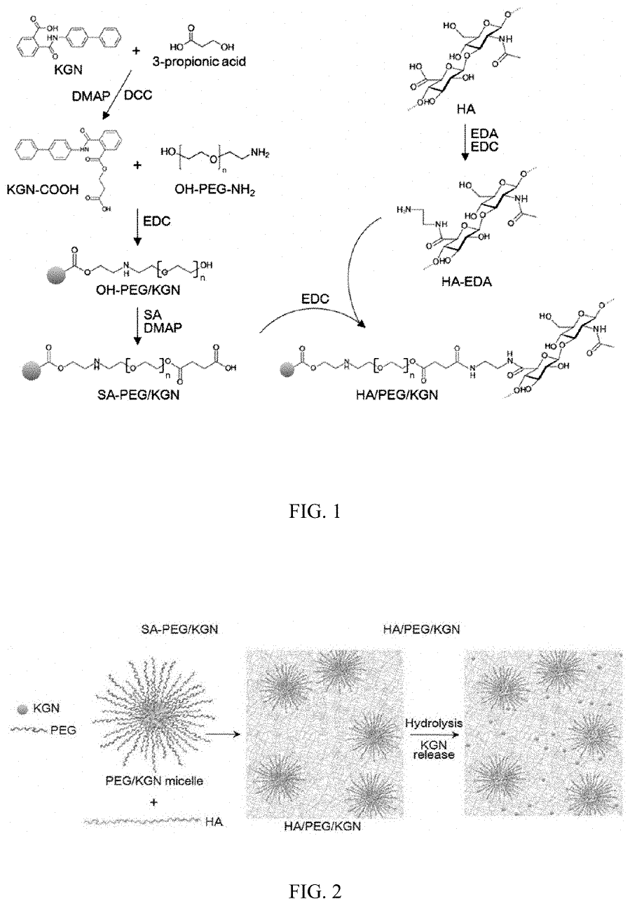

### DETAILED DESCRIPTION

Hereinafter, one or more specific exemplary embodiments will be described in more detail through Examples. However, these Examples are provided only for exemplarily explaining one or more specific exemplary embodiments, and the scope of the present invention is not limited by these Examples.

### Example 1. Confirmation of Production, Characteristics and Activity of Improving Osteoarthritis of Hyaluronic Acid-Polyethylene Glycol-Kartogenin Hydrogel

<1-1> Synthesis of PEGylated KGN

<1-1-1> Production of Ester Bond

Before the PEGylation was carried out on a kartogenin derivative, a relatively unstable ester bond was produced in kartogenin (KGN, MW=317.34 Da, Tocris Bioscience, Bristol, UK) by using 3-hydroxypropanoic acid.

Specifically, KGN (31.7 mg, 0.1 mmol in dimethyl sulfoxide (DMSO)) was dissolved in 10 mL of anhydrous CH₂Cl₂, the resulting solution was cooled to 0° C., and then 3-hydroxypropanoic acid (Toronto Research Chemicals, TRC, Toronto, Canada, a 30% aqueous solution, 30.2 μL, 0.1 mmol), dicyclohexylcarbodiimide (DCC, Sigma-Aldrich, St Louis, Mo., USA, 30 mg, 0.15 mmol), and 4-dimethylaminopyridine (DMAP, Sigma-Aldrich, St Louis, Mo., USA, 15 mg, 0.15 mmol) were added thereto. After the solution was stirred at 0° C. for 2 hours, stirred at room temperature (RT) for 72 hours, and filtered, the resulting material was precipitated three times with anhydrous diethyl ether, and then carboxylated KGN (COOH-KGN; a white powder) was obtained by drying the precipitate under vacuum.

<1-1-2> PEGylation

For PEGylation, the COOH-KGN produced in <1-1-1> was covalently bonded to a primary amine group of heterobifunctional O-(2-aminoethyl)polyethylene glycol (PEG; OH-PEG-NH₂, 2 kDa, Biochempeg, Watertown, Mass., USA) having two different functional groups by using 1-ethyl-3-(3-dimethylaiminopropyl)carbodiimide (EDC carbodiimide, Sigma-Aldrich, St Louis, Mo., USA).

Specifically, after COOH-KGN (0.01 mmol, 4.07 mg in DMSO) was dissolved in 10 mL of a 2-(N-morpholino)ethanesulfonic acid (MES) buffer containing EDC (19.17 mg, 0.1 mmol), OH-PEG-NH₂ (20 mg, 0.01 mmol) was added thereto. The reaction mixture was stirred gently for 24 hours, and then dialyzed against deionized water by using a Spectra/Por dialysis tube (molecular weight cut-off (MWCO)=2,000 Da)(Spectrum Lab., CA, USA). Next, the PEG/KGN was modified with a carboxyl group end by a method in the following <1-1-3>.

<1-1-3> Formation of Carboxyl Group End

In order to obtain a carboxylated PEG/KGN, the end hydroxyl group was modified with a carboxyl group by treating the PEG/KGN produced in <1-1-2> with succinic anhydride (SA, Toronto Research Chemicals, TRC, Toronto, Canada).

Specifically, after PEG/KGN (24.1 mg, 0.01 mmol), SA (1 mg, 0.01 mmol), DMAP (0.6% w/v), and triethylamine (0.01% v/v) were dissolved in anhydrous dioxane, the resulting solution was stirred at room temperature under nitrogen for 24 hours. Next, the solvent was removed by a rotary evaporator, and the residue was filtered, and then precipitated three times with ice-cold diethyl ether. Finally, a carboxylated PEG/KGN (white powder) was obtained by lyophilizing the precipitate under vacuum.

<1-2> Production of Micelle

A PEG/KGN micelle was produced by using dialysis technique.

Specifically, the carboxylated PEG/KGN (20 mg) produced and lyophilized in <1-1-3> and 4 pt of TEA were dissolved in 20 mL of DMSO. Thereafter, the solution was stirred at 80° C. for 24 hours, and then dialyzed against distilled water for 72 hours by using a tubular dialysis membrane (MWCO=3,000 Da). An aqueous H₂O₂ solution (1 mL, 3.0%) was added dropwise to the micelle solution under stirring and the resulting mixture was stirred for 3 hours, and then dialyzed against distilled water for 24 hours to remove an excessive amount of H₂O₂. A solid PEG/KGN micelle was recovered by adjusting the final concentration to 0.5 mg/mL and lyophilizing the dialyzed product.

<1-3> Synthesis of HA/PEG/KGN Hydrogel

After in vivo hydrolysis was reduced by transplanting ethylene diamine (EDA, Toronto Research Chemicals, TRC, Toronto, Canada) into a carboxyl group of hyaluronic acid (HA, sodium salt, MW 1,000 kDa, Sigma-Aldrich, St Louis, Mo., USA), a hyaluronic acid hydrogel containing the carboxylated PEG/KGN micelle produced in <1-2> was produced.

Specifically, HA (86.6 mg, 0.1 μmol) was dissolved in 100 mL of a MES buffer containing EDC (437.7 mg, 2.3 mmol), and EDA (13.7 mg, 0.23 mmol) was added thereto. Next, the reaction mixture was stirred gently for 24 hours, and then dialyzed against deionized water by the dialysis method in <1-1-2> to produce HA-EDA, and the product was lyophilized and used. Thereafter, a HA-EDA (2 wt %) hypotonic solution and the PEG/KGN micelle (2 wt %, 3.6 mmol of EDC in the hypotonic solution) produced in <1-2> and prepared in deionized water were mixed at a volume ratio of 1:1. Thereafter, the mixture was stirred gently for 24 hours, and then dialyzed against deionized water by the above-described dialysis method to produce a HA hydrogel containing a covalently-bonded PEG/KGN micelle.

Meanwhile, a HA hydrogel control containing no PEG/KGN micelle was produced by a method similar to that described above using HA-EDA (2 wt %).

<1-4> Confirmation of Critical Micelle Concentration of PEG/KGN

The critical micelle concentration of the PEG/KGN micelle produced in <1-2> was confirmed.

Specifically, the critical micelle concentration (CMC) of the PEG/KGN micelle was determined by a fluorescence spectrometer using pyrene (Sigma-Aldrich, St Louis, Mo., USA) as a fluorescent probe at an emission wavelength of 395 nm. Excitation spectra of 300 to 350 nm were recorded with a bandwidth of 5 nm. The CMC of the PEG/KGN micelle was determined by using an intensity ratio (I₃₃₇/I₃₃₄) of 337 to 334 nm.

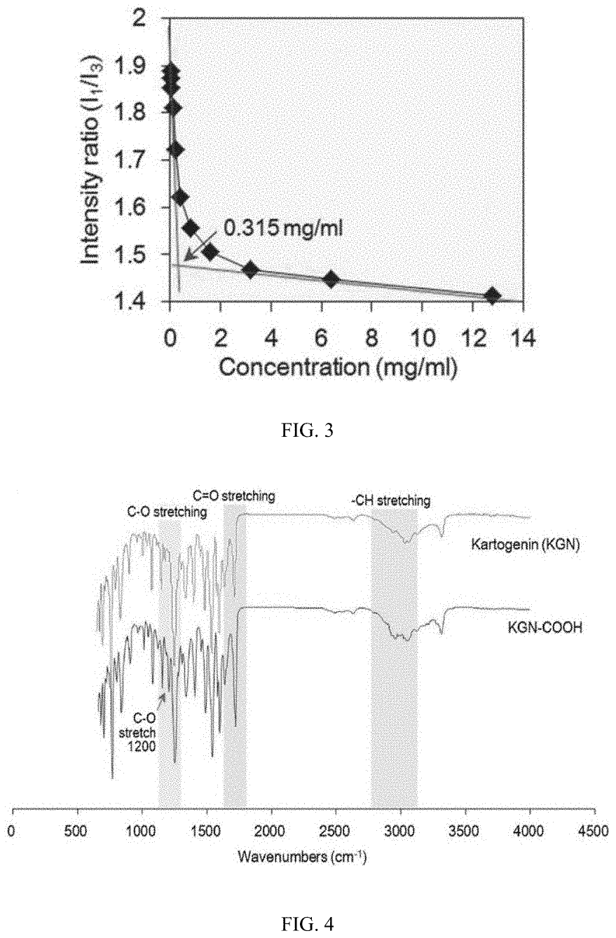

As a result, it was confirmed that the PEG/KGN micelle exhibited a low critical micelle concentration (CMC) of 0.0315 wt % (FIG. 3).

<1-5> Confirmation of Chemical Characteristics of PEG/KGN

Surface chemical characteristics of the PEG/KGN synthesized in <1-1-3> were confirmed by Fourier transform infrared spectroscopy (FTIR) and proton nuclear magnetic resonance spectroscopy (¹H NMR).

Specifically, the FTIR spectra were measured at room temperature within a range of 4,000 to 650 cm⁻¹ by using 32 scans and 8 cm⁻¹ resolution using a Nicolet 6700 FTIR spectrometer (Thermo Scientific). ¹H NMR was carried out by using Bruker Avance III 600 (600.13 MHz; Bruker BioSpin, Rheinstetten, Germany), and was exhibited as a chemical shift (δ) in parts per million (ppm) for deuterium (D₂O) or DMSO-d6.

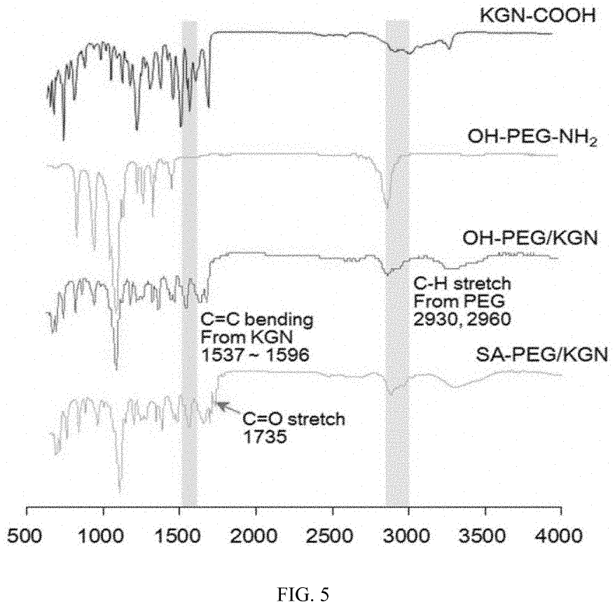

As a result, in <1-1-3>, the hydroxyl end of PEG/KGN was modified with a carboxyl group derived from the SA in order to induce a covalent crosslinking bond between HA-EDA and PEG/KGN, and the presence of the SA-PEG/KGN was confirmed by a 1735 cm⁻¹ peak of FTIR, that is, a vibration absorption peak (C═O, derived from SA) of a carbonyl group (FIG. 5).

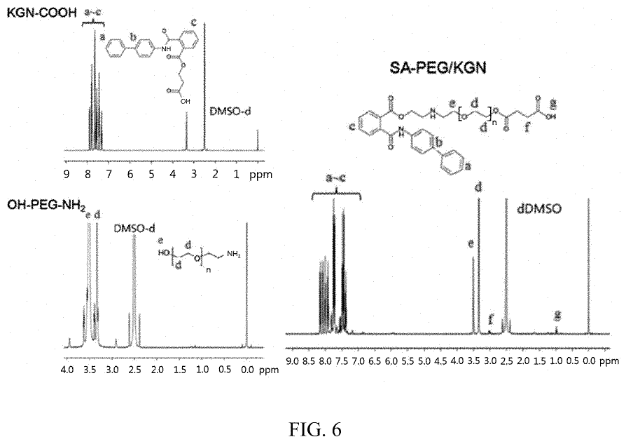

In the FTIR spectra of KGN—COOH, KGN to which 3-hydroxypropionic acid was bonded was confirmed by the presence of a C—O stretch peak at 1,200 cm⁻¹ (FIG. 4), and through FTIR (FIG. 5) and ¹H NMR (FIG. 6), it was confirmed that the PEG/KGN contained a hydrophilic PEG chain and a hydrophobic KGN residue.

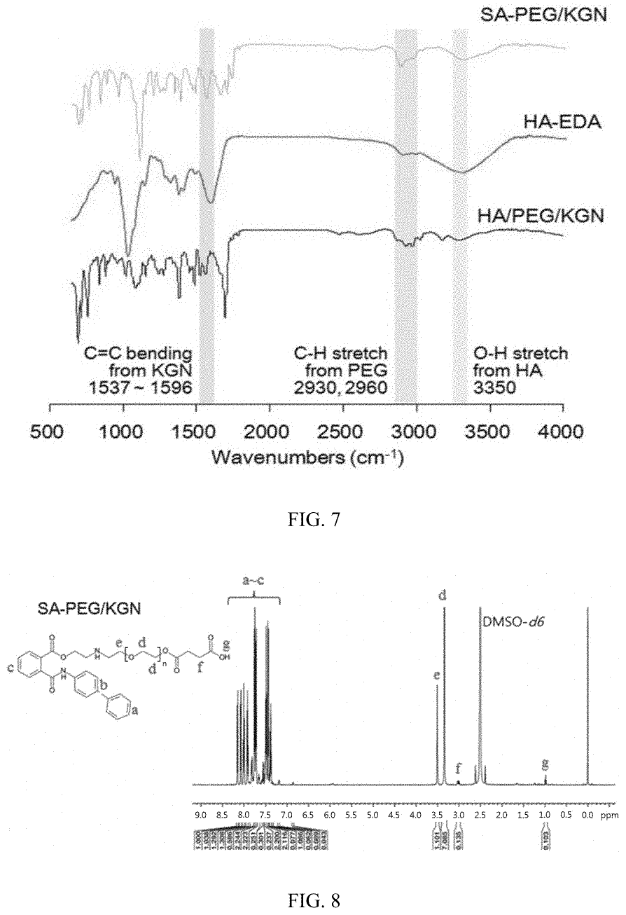

In the FTIR spectra of PEG/KGN, it was confirmed by a C═C bend peak (1,537 to 1,596 cm⁻¹) derived from an aromatic ring of KGN and a CH stretch peak (2,930 cm⁻¹) derived from PEG that KGN and PEG was successfully bonded to each other through the formation of an amide bond during the EDC catalyst process (FIG. 7).

In addition, in the ¹H NMR spectrum of PEG/KGN dissolved in DMSO-d6, characteristic chemical shifts corresponding to PEG (63.36 and 3.62 ppm) and KGN (87.3 to 7.9 ppm) were observed as sharp resonance peaks (FIG. 8).

<1-6> Confirmation of Chemical Characteristics of HA/PEG/KGN

Surface chemical characteristics of the HA/PEG/KGN synthesized in <1-3> were confirmed by Fourier transform infrared spectroscopy (FTIR) and proton nuclear magnetic resonance spectroscopy (¹H NMR).

Specifically, the FTIR spectra were measured at room temperature within a range of 4,000 to 650 cm⁻¹ by using 32 scans and 8 cm⁻¹ resolution using a Nicolet 6700 FTIR spectrometer (Thermo Scientific). ¹H NMR was carried out by using Bruker Avance III 600 (600.13 MHz; Bruker BioSpin, Rheinstetten, Germany), and was exhibited as a chemical shift (δ) in parts per million (ppm) for deuterium (D₂O) or DMSO-d6.

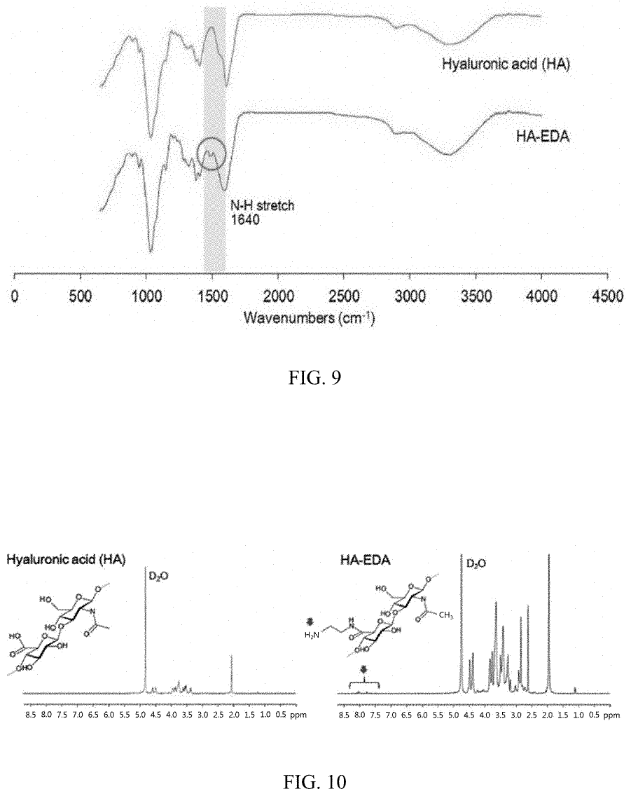

As a result, when HA was allowed to react with EDA before HA and PEG/KGN were crosslinked to each other, it was confirmed that the breakdown of HA was significantly reduced, and a covalent bond between HA and PEG/KGN was promoted. HA-EDA exhibited an N—H stretch peak at 1,640 cm⁻¹ by FTIR (FIG. 9) and exhibited a typical signal pattern at ˜δ7 of an amide NH proton by ¹H NMR (FIG. 10), so that it was confirmed that HA successfully formed an amine bond.

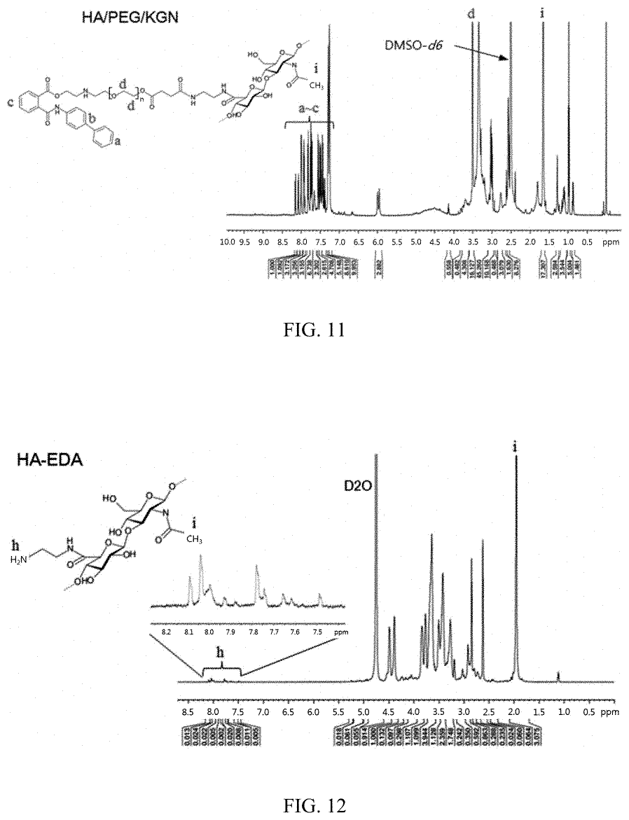

Further, the presence of PEG at HA/PEG/KGN was confirmed by the presence of a C—H stretch peak (2,930 and 2,960 cm⁻¹) derived from PEG (FIG. 7). By the presence of an aromatic peak (7.3 to 7.9 ppm) of KGN and a characteristic peak (1.9 ppm) which HA exhibited, it was confirmed that the bond of HA/PEG/KGN was successfully achieved (3H, —NH—CO—CH₃)(FIGS. 11 and 12).

That is, through the result, it was confirmed that a successful HA/PEG/KGN bond was formed through the formation of an amide bond in the EDC catalyst process.

<1-7> Confirmation of Micelle Morphology of HA/PEG/KGN Hydrogel

In order to observe the micelle morphologies of the HA free PEG/KGN micelle and the HA/PEG/KGN hydrogel produced in <1-3>, scanning electron microscopy (FE-SEM; ZEISS SUPRA 53VP, Carl Zeiss AG, Oberkochen, Germany) and light-scattering spectrophotometry were carried out.

Specifically, one drop of each aqueous PEG/KGN or HA/PEG/KGN dispersion was placed on a stud and allowed to dry. After HA/PEG/KGN was subjected to SEM so as to be) maximally expanded in water at room temperature for 24 hours, the HA/PEG/KGN hydrogel was rapidly lyophilized so as to maintain the original form.

Meanwhile, the lyophilized HA/PEG/KGN hydrogel sample was cut off and fixed on a stub, and all the samples were coated with gold in order to observe the internal morphology thereof. The particle size distribution of the PEG/KGN micelle included in HA and the covalently-bonded PEG/KGN micelle was determined by using a dynamic light scattering spectrophotometer (DLS, Otsuka Electronics Ltd., Osaka, Japan) equipped with argon ion laser (488 nm) at 25° C., the scattering angle was set at 90° C., and the result was measured at least three times and indicated as means±SD.

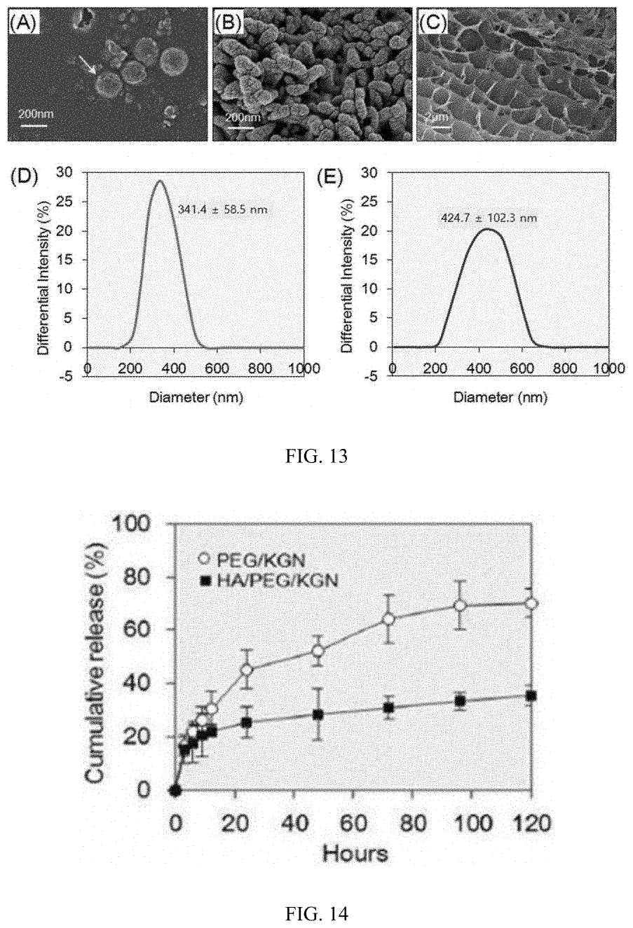

As a result, the presence of the PEG/KGN micelle in water was confirmed by SEM. FIG. 13A illustrates an individual micelle with a dark core and a bright shell. It was confirmed that the PEG/KGN micelle covalently bonded to HA was differentiated from an irregular elliptical shape, and had a more rigid core with a harder surface than the PEG/KGN micelle (FIG. 13B).

Meanwhile, the lyophilized HA/PEG/KGN hydrogel exhibited the formation of irregular pores less than 5 μm on the SEM image. Since the lyophilization causes HA/PEG/KGN to collapse and form pores in the hydrogel matrix, it was confirmed that the morphology of the lyophilized hydrogel matrix was different from the matrix derived from the air drying condition (FIG. 13).

Furthermore, as a result of the DLS analysis of the PEG/KGN micelle, a hydrodynamic diameter of 341.44±58.5 nm (polydispersity index 0.214±0.005) was confirmed (FIG. 13D), and after the crosslinking in the HA hydrogel, it was confirmed that the average PEG/KGN micelle diameter was increased to 424.74±102.3 nm (polydispersity index 0.431±0.012)(FIG. 13E). It was confirmed that the reason that the particle sizes measured by the SEM were smaller than the hydrodynamic size measured by the DLS analysis was because the DLS measures the particle sizes in water whereas the SEM provides dimensions in a dry state.

<1-8> Confirmation of In Vitro Release Activity of KGN

The in vitro KGN release activity of the PEG/KGN micelle produced in <1-2> and the HA/PEG/KGN hydrogel produced in <1-3> was confirmed.

Specifically, the PEG/KGN micelle containing about 10 mg of KGN prepared in <1-2> or the HA/PEG/KGN hydrogel produced in <1-3> were incubated in a simulated body fluid (SBF, pH 7.8) at 37° C. while being stirred gently (90 rpm). After centrifugation (14,000 g, 10 minutes), SBF was collected, and was replaced with a fresh SBF at each sampling time. The content of KGN in the collected buffer was measured by reverse-phase high-performance liquid chromatography (HPLC, Ultimate 3000, Thermo Dionex, Sunnyvale, Calif., USA) using an Inno C-18 column (150×4.6 mm, 5 μm, Youngjinbiochrom, Seoul, Korea). An analysis was carried out under an isocratic condition at a flow rate of 1.0 mL/min. The chromatogram was recorded at 274 nm, and the calibration curve for KGN was linear within a range of 1 to 100 mg/L.

As a result, an in vitro release test was carried out statically under a sink condition, KGN was rapidly released from the PEG/KGN micelle, and 51.2±5.7% was cumulatively lost within 48 hours. In contrast, when KGN was released from the HA/PEG/KGN hydrogel, an initial burst over 12 hours followed by a sustained release over 5 days and a cumulative loss of 32.4±3.3% occurred (FIG. 14), confirming that the reason was because the release of KGN from a hydrophobic core was delayed by physical encapsulation of the PDG/KGN micelle in the HA hydrogel.

Meanwhile, it was confirmed that KGN was released by diffusion into the HA hydrogel matrix through the PEG layer and diffusion into an external medium through the hydrogel, and a covalent bond formed between PEG and the HA matrix further delayed the release of KGN.

<1-9> Confirmation of In Vitro Breakdown of HA/PEG/KGN Hydrogel by Enzyme

A synthetic biomaterial needs to be able to be enzymatically broken down when used in the regeneration of tissues. Accordingly, in order to confirm whether the HA/PEG/KGN hydrogel was broken down by an enzyme, it was confirmed whether HA/PEG/KGN was broken down in vitro by treating the HA/PEG/KGN hydrogel produced in <1-3> with collagenase and hyaluronidase (HAase).

Specifically, the HA/PEG/KGN hydrogel was incubated in 1 mL of PBS containing 5 U/ml of collagenase and/or HAase, and incubated at 150 rpm at 37° C. The buffer used in collagenase was a 100 mM Tris-HCl buffer (pH 7.4) containing 5 mM CaCl₂ and 0.05 mg/ml sodium azide. The breakdown of HAase was carried out in 30 mM citric acid, 150 mM Na₂HPO₄, and 150 mM NaCl (pH 6.3). A 100 mM Tris-HCl buffer (pH 7.4) containing 5 mM CaCl₂, 150 mM NaCl, and 0.05 mg/ml sodium azide was used in the simultaneous breakdown of collagenase and HAase. The supernatant was sucked out every two days, and the breakdown medium was replenished with a fresh enzyme solution The mass loss fraction was determined by a mathematic formula of Wₜ/W₀×100(%), and here, Wₜ denotes a weight of HA/PEG/KGN at the time t, and W₀ denotes an initial weight of HA/PEG/KGN.

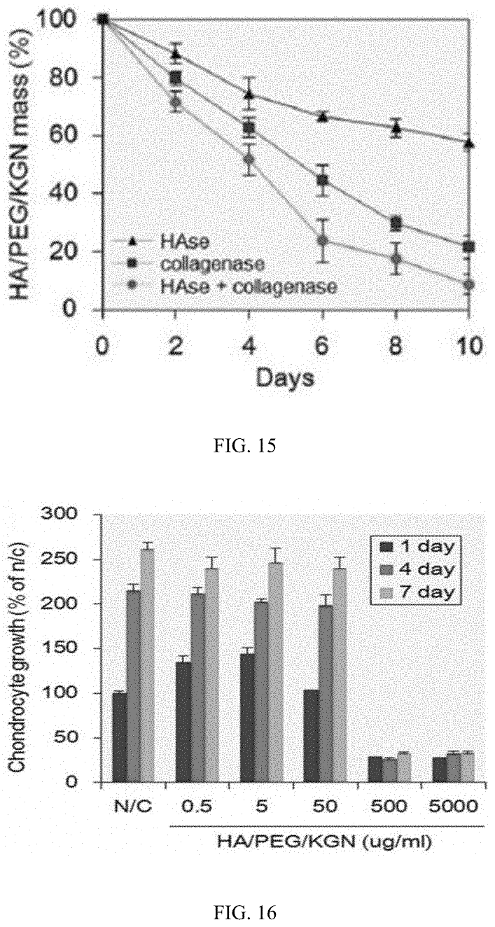

As a result, HA/PEG/KGN was slowly broken down by HAase for 10 days, and as a result, 42.84±2.7% of the mass was reduced. Meanwhile, the breakdown of HA/PEG/KGN by collagenase was more rapid than that by HAase, and when the HA/PEG/KGN hydrogel was treated with collagenase and HAase, the HA/PEG/KGN was reduced by 90% or more within 10 days (FIG. 15).

That is, through the result, it was confirmed that the HA/PEG/KGN hydrogel could withstand HAase better than collagenase.

### Example 2. Confirmation of Activity of Improving Osteoarthritis of Hyaluronic Acid-Polyethylene Glycol-Kartogenin Hydrogel

<2-1> Preparation and Culture of Cells

Bone marrow-derived mesenchymal stem cells (BMSCs) were separated from bone marrow samples obtained from three patients with degenerative arthritis (average age: 64 years old, range: 54 to 72 years old), who were subjected to total hip replacement, chondrocytes were separated from the fragments of human articular cartilage (AC) obtained from three patients with degenerative arthritis (average age: 62 years old, range: 59 to 65 years old), who were subjected to total knee arthroplasty, and written informed consents were obtained from all the donors. It was confirmed that characteristics of the separated BMSCs were the same as the previously known BMSC flow cytometric analysis results.

Cells for use in the evaluation of anti-osteoarthritic activity were cultured by using a Dulbecco's modified Eagle's medium/F-12 (DMEM/F-12, Gibco, Grand Island, N.Y., USA) and a bovine serum albumin (BSA, Gibco, Grand Island, N.Y., USA).

<2-2> Preparation of Animal Model

Animal experiments were performed with the approval of the animal experiment ethics committee by using 9-week old male Sprague Dawley rats (Orient Inc., Seoul, Korea). Degenerative arthritis (osteoarthritis; OA) was induced by performing anterior cruciate ligament transection (ACLT) and medial meniscectomy (MM) on the rats, and the rats were allowed to exercise on a treadmill for 20 minutes every day from two weeks after the surgery.

<2-3> Statistical Analysis

In the Examples of the present invention, technical statistics were used in order to determine the group average and standard deviation. The Group OARSI and Mankin scores were compared by using an one-way analysis of variance (SPSS 15.0; SPSS Inc., IL, USA) using post-hoc analyses of Nonparametric (Mann Whitney U test) and Bonferroni, and a P value less than 0.05 was considered to indicate a statistical significance.

<2-4> Confirmation of Cytotoxicity Range of HA/PEG/KGN Hydrogel

The cytotoxicity of the HA/PEG/KGN hydrogel produced in <1-3> was investigated through an MTT analysis.

Specifically, after the HA/PEG/KGN hydrogel was treated with the chondrocytes produced in <2-1> at various concentrations (0, 0.5, 50, 500, and 5,000 μg/ml), the MTT analysis was carried out for 7 days. After an MTT reagent (tetrazolium salt solution) was added directly to cells containing the HA/PEG/KGN hydrogel (per solution, macromere n=3), and then allowed to react in an incubator at 37° C. for 4 hours, the purple formazan produced by active mitochondria was solubilized in DMSO with orbital shaking for 2 hours. The absorbance of these solutions was read at 570 nm (SpectraMax 384, Molecular Devices, Sunnyvale, Calif., USA).

Meanwhile, in order to evaluate the cytotoxicity and viability, Live/Dead analysis was performed by using a Live/Dead fluorescent staining kit (Invitrogen, Carlsbad, Calif., USA) and the BMSC prepared in <2-1>, and after the HA/PEG/KGN hydrogel (50 μg/ml) was treated with BMSC for 7 days, live (green) cells and dead (red) cells were observed by incubation with a staining reagent for 30 minutes and washing with PBS.

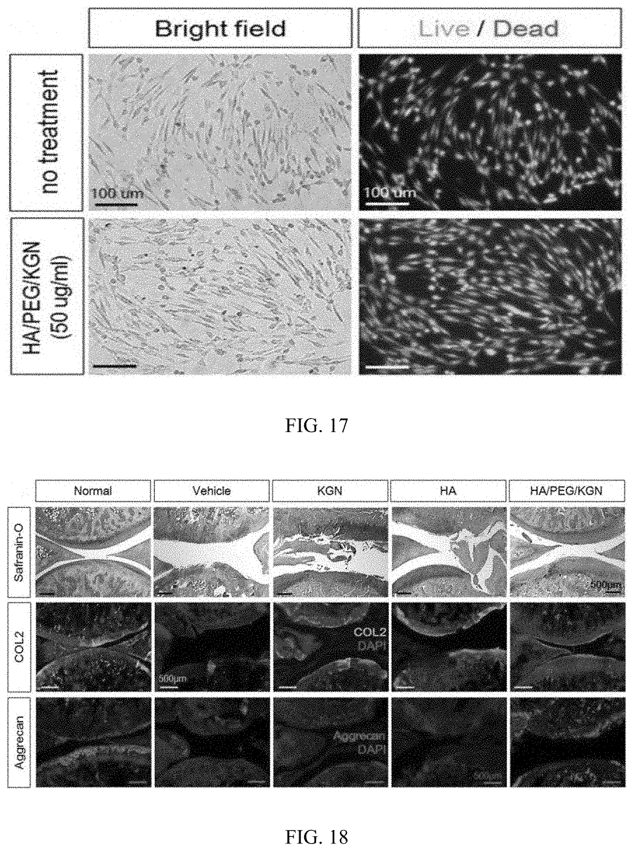

As a result, when HA/PEG/KGN was treated at a concentration of 50 μg/ml or less, it was confirmed that the proliferation of cells was maintained well for 7 days (FIG. 16), and through the Live/Dead analysis result, it was confirmed that the cells were living (FIG. 17). In contrast, it was confirmed that at a concentration exceeding 500 μg/ml, the proliferation of chondrocytes was reduced, and concentration-dependent cytotoxicity occurred.

<2-5> Confirmation of In Vivo Effect of HA/PEG/KGN Hydrogel

The effect of regenerating the intra-articular (IA) chondrocytes to which the HA/PEG/KGN hydrogel produced in <1-3> was administered was evaluated by using rats in which OA was induced in <2-2>.

Specifically, the HA/PEG/KGN hydrogel (50 mg in 100 μL of PBS) was injected into the knee joint of the rat with OA twice at 7th week and 10th week after the surgery. The rats were treated with IA injection of a free HA hydrogel control (50 mg in 100 μL of PBS) by the same method, and 100 μM KGN in 100 μL of PBS or a vehicle (100 μL PBS) control was injected by the same method.

The rats were sacrificed at 8th week after the first IA injection, the knee joints were dissected and fixed with 10% paraformaldehyde (at 4° C. for 1 day), and then calcareous materials were removed with a Lite decalcifying solution (Sigma-Aldrich), and after the resulting knee joints were embedded in a Tissue-Tek OCT compound (Sakura Finetek, Torrance, Calif., USA) or paraffin wax, and then the paraffin wax fragment was stained with Safranin-O (4% w/v) and Fast Green (0.1% w/w). The degenerative state was evaluated by using an Osteoarthritis Research Society International (OARSI) cartilage Histopathology Assessment System and a Mankin evaluation system, and the fragments were analyzed through immunohistochemistry for COL2 and aggrecan. As a primary antibody, a mouse anti-COL2AI monoclonal antibody (Millipore; 1/100) or a rabbit anti-aggrecan polyclonal antibody (Abcam, Cambridge, UK; 1/100) was used.

As a result, it was confirmed that in the rats treated with the vehicle, extensive cartilage destruction occurred due to the removal of matrix loss and surface defects, and in the rats treated with a soluble KGN or a free HA hydrogel, there is a loss of cartilage due to matrix vertical cracks and surface layer exfoliation. However, in the case of the rats which was injected with the HA/PEG/KGN hydrogel, it was confirmed that the destabilization of the surface was slight and the cartilage was further thickened (FIG. 18). That is, through the results, an excellent cartilage protection effect of the HA/PEG/KGN hydrogel was confirmed as compared to the free HA hydrogel.

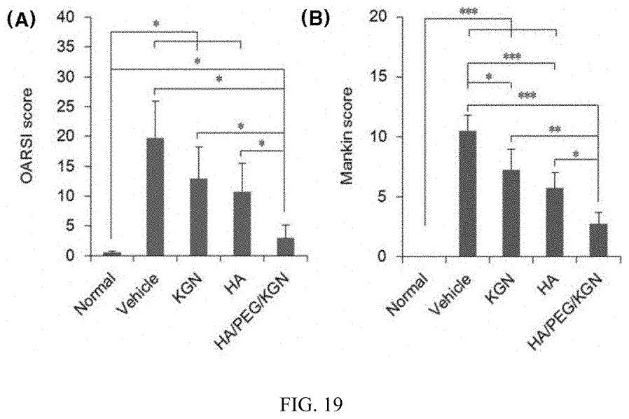

Meanwhile, as a result of performing an immunohistochemical analysis for COL2 and aggrecan in order to evaluate the biochemical change of the human articular cartilage composition, it was confirmed that in the rats injected with the HA/PEG/KGN hydrogel, strong COL2 and aggrecan staining occurred, and the occurrence was in a level similar to that of a normal control, but in the cartilage of the rats treated with a vehicle, this staining was not observed (FIG. 18). Further, it was confirmed that the OARSI and Mankin scores of the rats injected with the HA/PEG/KGN hydrogel were significantly lower than those of mice injected with a free HA gel (FIGS. 19A and 19B).

That is, through the results, it was confirmed that HA/PEG/KGN had excellent cartilage regeneration effects.

From the foregoing, the present invention has been reviewed mainly based on the preferred examples thereof. A person with ordinary skill in the art to which the present invention pertains will be able to understand that the present invention may be implemented in a modified form without departing from the essential characteristics of the present invention. Therefore, the disclosed examples should be considered not from a restrictive viewpoint, but from an explanatory viewpoint. The scope of the present invention is defined not in the above-described explanation, but in the claims, and it should be interpreted that all the differences within a range equivalent thereto are included in the present invention.

What is claimed is:

1. A polymeric micelle comprising a PEGylation product of a polyethylene glycol derivative and a kartogenin derivative,
wherein the polyethylene glycol derivative is a polyethylene glycol derivative in which one of the end hydroxyl groups of polyethylene glycol is substituted with an amine group.

2. The polymeric micelle of claim 1, wherein the polyethylene glycol derivative is a compound represented by the following Formula 1: 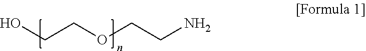
(n is an integer from 1 to 100).

3. The polymeric micelle of claim 1, wherein the PEGylation product is a compound represented by the following Formula 2: 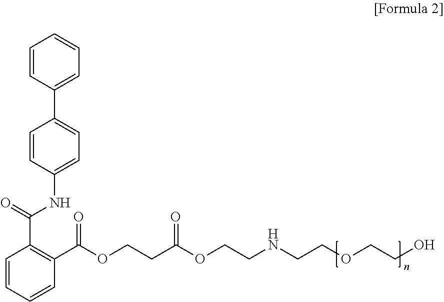
(n is an integer from 1 to 100).

4. The polymeric micelle of claim 1, wherein the PEGylation product is a compound represented by the following Formula 3: 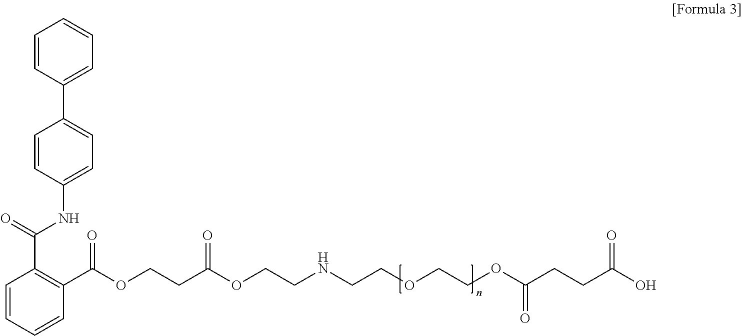
(n is an integer from 1 to 100).

5. A hyaluronic acid derivative hydrogel, wherein the hydrogel comprises the polymeric micelle of claim 1, and
the hyaluronic acid derivative is formed by condensation of a carboxyl group of hyaluronic acid with an amine group of alkylene diamine.

6. The hyaluronic acid derivative hydrogel of claim 5, wherein the alkylene diamine is ethylene diamine.

7. The hyaluronic acid derivative hydrogel of claim 5, wherein the polymeric micelle contains a compound represented by the following Formula 2.

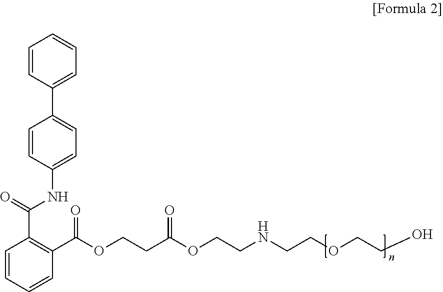

8. The hyaluronic acid derivative hydrogel of claim 5, wherein the polymeric micelle contains a compound represented by the following Formula 3.

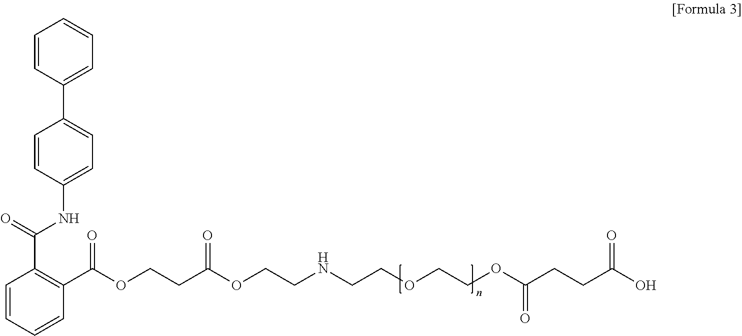

9. The hyaluronic acid derivative hydrogel of claim 5, wherein the hyaluronic acid derivative is a compound represented by the following Formula 4: 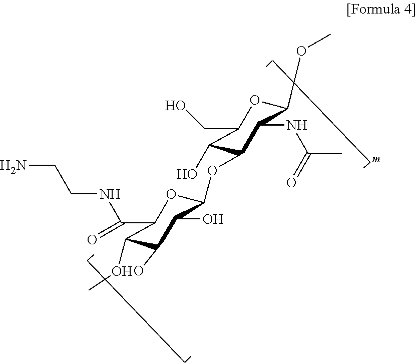
(m is an integer from 100 to 3,000).

10. The hyaluronic acid derivative hydrogel of claim 5, wherein the polymeric micelle is completely or partially covalently bonded to the hyaluronic acid derivative.

11. The hyaluronic acid derivative hydrogel of claim 10, wherein the covalent bond is formed by condensation of a carboxyl group derived from the polymeric micelle with an amine group derived from the hyaluronic acid derivative.

12. The hyaluronic acid derivative hydrogel of claim 11, wherein the hyaluronic acid derivative hydrogel comprises a compound represented by the following Formula 5: 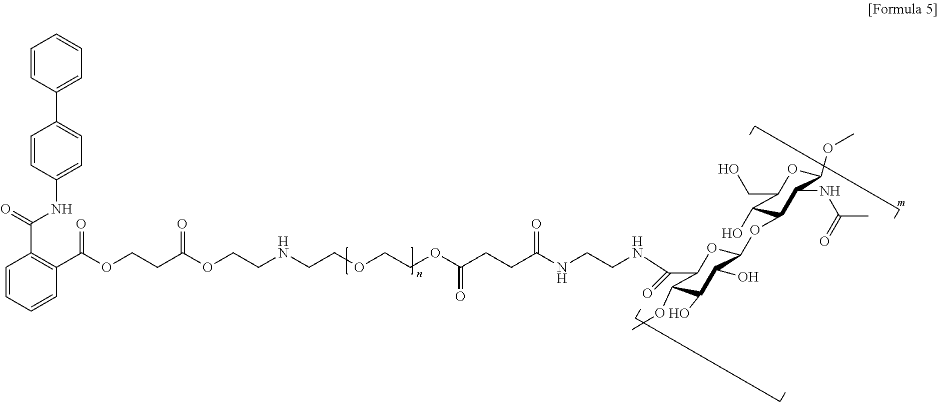
(n is an integer from 1 to 100, and m is an integer from 100 to 3,000).

13. A composition for protecting or regenerating cartilage, comprising the hyaluronic acid derivative hydrogel of claim 5.

14. The composition of claim 13, wherein the hyaluronic acid derivative hydrogel contains a compound represented by the following Formula 5.

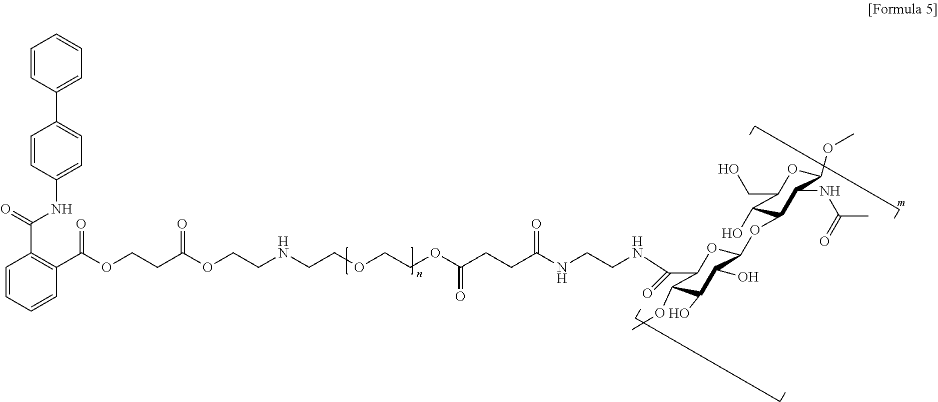

15. A pharmaceutical composition for preventing or treating degenerative arthritis, comprising the hyaluronic acid derivative hydrogel of claim 5.

16. The pharmaceutical composition of claim 15, wherein the hyaluronic acid derivative hydrogel contains a compound represented by the following Formula 5.

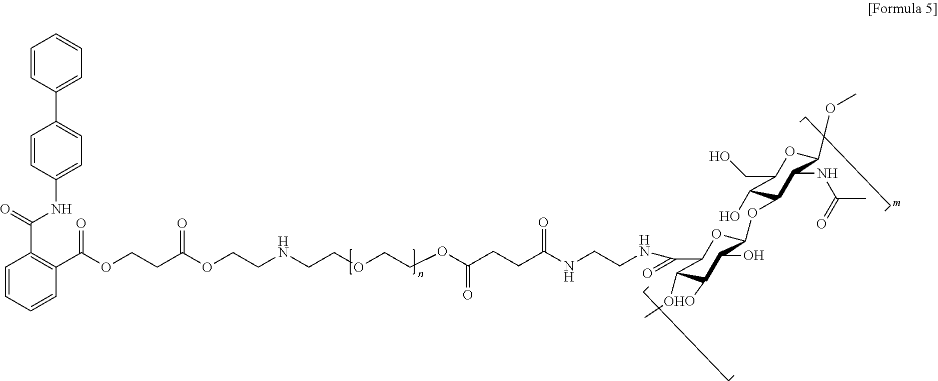

17. The pharmaceutical composition of claim 15, wherein the composition is an injectable dosage form.

18. A method for producing a polymeric micelle, the method comprising:
producing a compound represented by the following Formula 8, in which an ester group is formed by allowing kartogenin represented by the following Formula 6 and 3-hydroxypropionic acid represented by the following Formula 7 to react with each other; 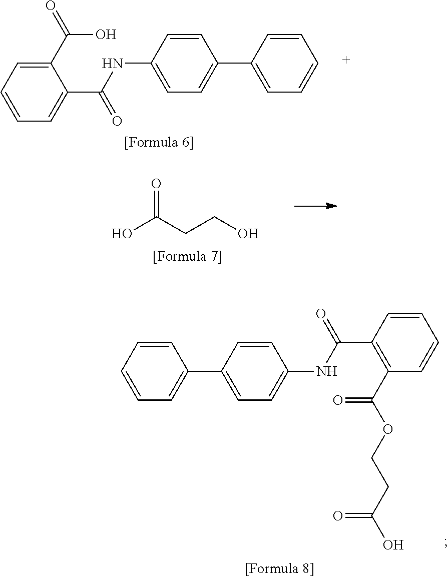 [SMILES: O=C(O)c1ccccc1C(=O)Nc1ccc(-c2ccccc2)cc1 ; O=C(O)CCO ; O=C(O)CCOC(=O)c1ccccc1C(=O)Nc1ccc(-c2ccccc2)cc1]
producing a compound represented by the following Formula 2, in which a secondary amine group is formed by allowing a compound represented by the following Formula 8 and a compound represented by the following Formula 1 to react with each other 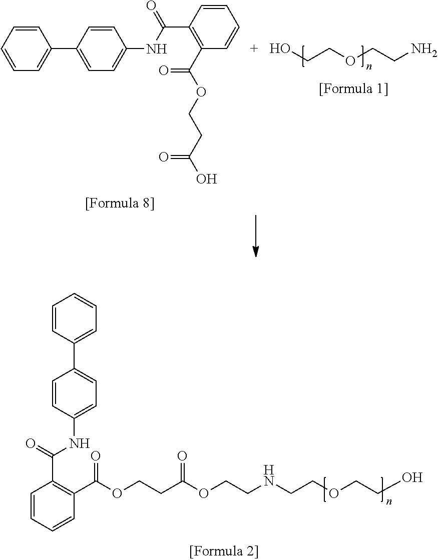
(n is an integer from 1 to 100); and
producing a compound represented by the following Formula 3, into which a carboxyl group is introduced by allowing a compound represented by the following Formula 2 and succinic anhydride to react with each other 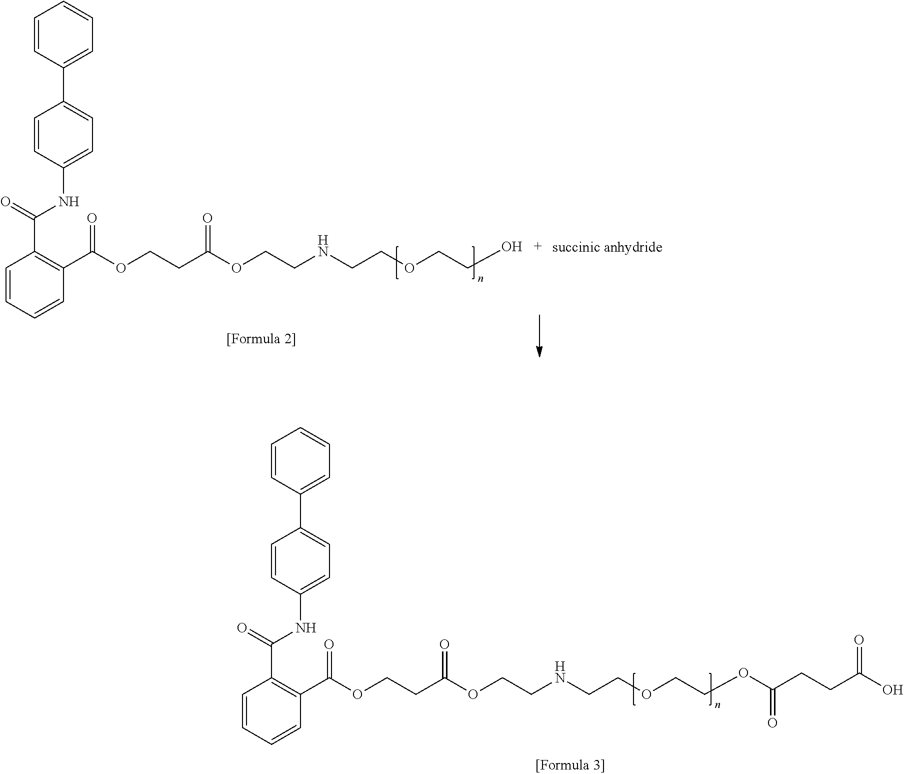
(n is an integer from 1 to 100).

19. A method for producing a hyaluronic acid derivative hydrogel, the method comprising:
producing a hyaluronic acid derivative represented by the following Formula 4, in which an amide group is formed by allowing hyaluronic acid represented by the following Formula 9 and ethylene diamine to react with each other 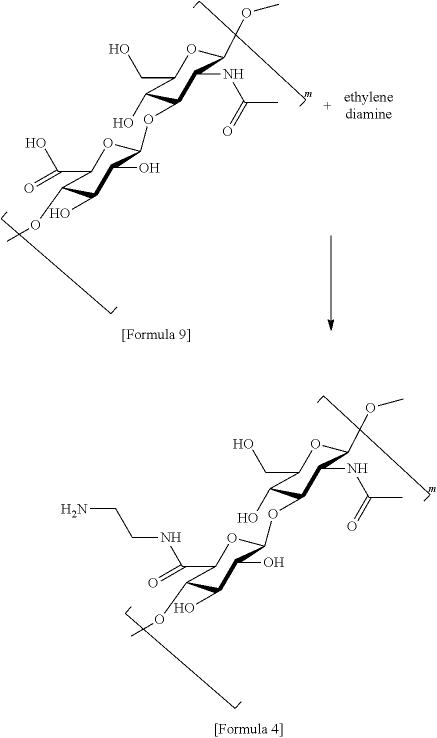
(m is an integer from 100 to 3,000); and
producing a hyaluronic acid derivative hydrogel comprising a compound represented by the following Formula 5 by allowing the polymeric micelle produced by the production method of claim 18 and the compound represented by Formula 4 to react with each other 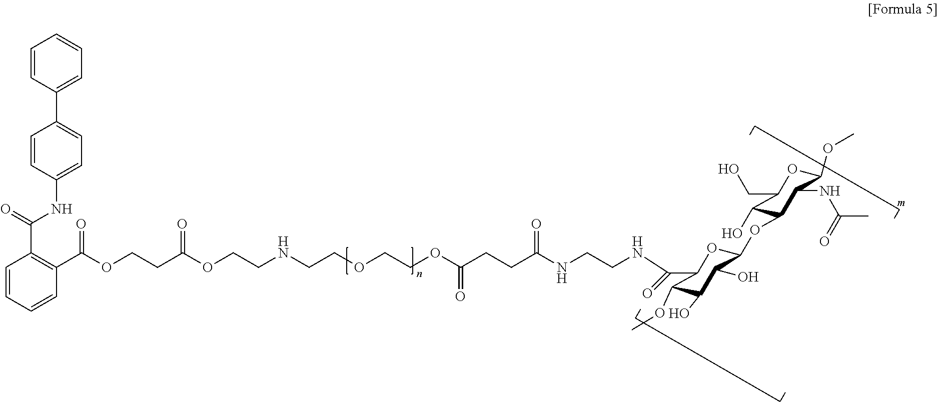
(n is an integer from 1 to 100, and m is an integer from 100 to 3,000).
